(19) United States
(12) Patent Application Publication
(10) Pub. No.: US 2009/0238796 A1
(43) Pub. Date: Sep. 24, 2009

(54) Preparation for treatment of non-infectious inflammatory intestinal diseases

(76) Inventors: Tamara Vasilevna Maculevich, (US); Valeriy Dmitrievich Bolotov, (US); Ekaterina Olegovna Doroshenko, (US); Igor Izmailovich Vaynshtok, (US)

Correspondence Address: Lauson & Tarver LLP, 880 Apollo Street, SUITE 301, El Segundo, CA, 90245, US

(21) Appl. No.: 12/077,991

(22) Filed: Mar. 24, 2008

(51) Int. Cl. A61K 35/74; A61P 1/00

(52) U.S. Cl. 424/93.3; 424/93.4; 424/93.45

(57) ABSTRACT

The invention relates to medicine and can be used for treating non-infectious inflammatory intestinal diseases to enhance efficiency of the non-infectious inflammatory intestinal disease treatment and reduce the length, the inventive preparation contains 5-aminosalicylic acid (5 ASA) or a substance, which decomposes in the organism that 5 ASA is formed, and a liophylically dried microbial mass of live bifido- or lactobacteria or the mixture, said bacteria can be immobilised on a sorbent. The 5 ASA can be powdered or granulated mesalazine or sulphasalazine. To ease the administration of preparation and ensure the dilution of components in defined sectors of the gastrointestinal tract, said components can be grouped in two capsules that jointly represent a single dose, wherein one capsule contains a microbial mass of live bacteria and the other capsule contains mesalazine or sulphasalazine or the capsule containing microbial mass of live bacteria is arranged in the mesalazine or sulphasalazine-containing capsule.

The invention pertains to medicine and can be used for treatment of non-infectious inflammatory intestinal diseases.

Non-infectious inflammatory intestinal diseases, ulcerative colitis and Crohn's disease among them, have been, and still are, a most serious problem in modern gastroenterology. The worldwide incidence of ulcerative colitis and Crohn's disease increases every year, and mainly among able-bodied population, which makes them socially significant diseases.

The similarity between these digestive tract lesions is that at their core there are chronic lesions of mucous membrane; and the clinical course proceeds in phases, with exacerbation periods and remission stages. So far, the actual ethiopathogenic reason for the onset of both inflammatory processes and their transition to a chronic form has not been found. On the other hand, the diseases differ substantially in terms of macroscopic histological pattern, as well as the localization site in the digestive tract.

In the case of ulcerative colitis, inflammation captures solely the large intestine mucous membrane, propagating successively from ist distal to proximal portion and pervading the entire large intestine, including the rectum, in hard cases.

In the case of Crohn's disease, inflammation process can affect any part, from mouth to anus. With this disease, the most often occurrence is combined lesion of lower portions of the small and large intestine, with segmented localization of lesions, when, next to an affected area, one finds areas with unchanged mucous membrane.

For treatment of non-infectious inflammatory intestinal diseases one uses sulfasalazane and mesalazane—preparations of 5-aminosalicylic acid.

Preparation sulfasalazane is known (Drug Encyclopedia. Register of Pharmaceuticals of Russia PJIC, Moscow, 000 RLS-2005, 2004,12th edition, p. 826-827). It has anti-bacterial and anti-inflammatory action. In the connective tissue of the intestine it breaks down into 5-aminosalicylic acid (5-ASA) responsible for anti-inflammation properties of sulfasalazane, and sulfapyridine, a competitive antagonist of paraaminobenzoic acid, which stops the synthesis of folates in microorganism cells and is responsible for anti-bacterial activity. The preparation shortcoming in treatment of ulcerative colitis and Crohn's disease is the strong dependence on the condition of the intestine normoflora which takes part in biotransformation of sulfasalazane and the release of 5-ASA, the active component, while sulfapyridine, the second component of the preparation, suppresses bacteria and actually inhibits and stops specific activity of the preparation. Possibly, this is the reason for a sharp increase in allergic developments and for liver function disturbance.

Also known is preparation salophalk—tablets covered with a shell soluble in the intestine and containing mesalazane—5-aminosalicylic acid (Drug Encyclopedia. Register of Pharmaceuticals of Russia PJIC, Moscow, 000 RLS-2005, 2004, 12th edition, pp. 780-781). The preparation is an anti-inflammatory agent that inhibits the synthesis of metabolites of arachidonic acid and the activity of neutrophilic lipoxygenase, inhibits migration, neutrophil degranulation and phagocytosis, and lymphocytes secretion of hemoglobulins, and binds and destroys free oxygen radicals. It is indicated for treatment of ulcerative colitis and Crohn's disease. Among the preparation shortcomings are acute allergic developments and disorders of the intestine function and nervous, cardiovascular and urinary systems, claimed as side effects. Hence, recommendations to use the preparation only under physician's supervision and have regular blood tests and urinalyses.

Also known is a pharmaceutical composition for oral administration that contains 5-aminosalicylic acid (5-ASA) as an active ingredient and is adapted for modified release from the shell in order to derive a clinically important localized effective profile of 5-ASA by releasing the corresponding amount of 5-ASA into the small and large intestine (patent RF JNk 2181043 C2, MI [symbol] 7 A61K 9/22, A61K 9/16, A61K 1/606, A61P 1/00, published 10.04.2002). Some of the shortcomings are as follows. For 5-ASA to demonstrate pharmacological activity a sufficiently active condition of normoflora is necessary along with general detoxifying action, normoflora taking part in the reaction of 5-ASA acetylation and excretion from the intestine. However, the general condition of normoflora in ulcerative colitis and Crohn's disease and especially of its anaerobic section (bifidobacteria and lactobacteria) is sharply inhibited even prior to the start of a specific treatment, and must be corrected, especially during periods of acute attack of the disease.

The invention objective is to create a preparation for treatment of inflammatory intestinal diseases that has no above mentioned shortcomings and that also contains bacteria that are characteristic for normal microflora of the gastrointestinal tract.

The technical result of the invention is higher efficiency of treatment of non-infectious inflammatory intestinal diseases and shorter length of treatment.

The technical result is achieved due to the fact that the preparation for treatment of inflammatory intestinal diseases that contains 5-aminosalicylic acid or a substance that breaks down in the body and forms 5-aminosalicylic acid, in addition also contains a liophylically dried microbial mass of live bifidobacteria or lactobacteria, or a mixture thereof, with the following component ratio, g: 5-aminosalicylic acid or a substance that breaks down in the body and forms 5-aminosalicylic acid 0.05-1.00 a liophylically dried microbial mass of live bacteria 10⁵-10¹⁰ CFU. The preparation contains bifidobacteria or bifidobacteria immobilized on a sorbent.

The preparation contains lactobacteria or lactobacteria immobilized on a sorbent. The preparation contains a mixture of bifidobacteria and lactobacteria, or a mixture of bifidobacteria and lactobacteria immobilized on a sorbent.

As 5-aminosalicylic acid or a substance that breaks down in the body and forms 5-aminosalicylic acid, the preparation contains mesalazane or sulfasalazane. Mesalazane or sulfasalazane are in the form of powder or granules.

The preparation components are grouped into two capsules that together form a single dose, wherein one capsule contains a microbial mass of live bacteria, and the other capsule contains mesalazane or sulfasalazane.

Alternately, the components are grouped into two capsules, wherein one capsule contains a microbial mass of live bacteria and is located inside the second capsule that contains mesalazane or sulfasalazane. The essence of the invention is as follows. There are numerous theories and assumptions about the causes of onset of non-infectious inflammatory intestinal diseases, in particular, disturbances of intra-intestinal immune system; autoimmune processes; genetic susceptibility to action of microbial toxins or to inadequate immune response, etc.

The information available in recent years, as well as our research, indicate that patients with ulcerative colitis and Crohn's disease have deep microenvironment disturbances in their digestive tract which makes it possible to assume that at the core of these intestinal diseases is imbalance of ecosystem “host—host's microflora”. This can also be corroborated by observations of substantial improvement in patients' condition when they are prescribed specially selected probiotics that normalize the composition of intestinal microflora. Microenvironment analysis of contents of the lower part of small intestine in cases of chronic inflammatory lesions of the digestive tract finds increased proportion of potentially pathogenic aerobic enterobacteria, bacteroids and other anaerobic gram-negative bacteria, and gram-positive aerobic and anaerobic cocci. A similar pattern is also found in studies of contents of the large intestine. A fundamentally important indicator of reduced colonization resistance of the digestive tract in cases of chronic intestinal diseases is a sharp decrease of the number of bifidoflora and lactobacilli, natural antagonists of potentially pathogenic microorganisms, in contents of the digestive tract, and increased proportion of bacteroids, anaerobic gram-positive coccobacilli and aerobic enterobacteria. The ratio of the number of anaerobes to aerobes in parietal microflora drops sharply, to as low as 25:1.

The quantitative content of bacteroids, bifidobacteria, eubacteria, peptococci, streptococci and enterobacteria, as well as organic acids, in feces of people with ulcerative colitis, is substantially different for sick and healthy people. Comparative studies of content of lactobacilli, bifidobacteria and bacteroids in feces of healthy people and people with Crohn's disease during exacerbation have demonstrated sharp reduction of the number of bifidobacteria in sick people. Reduction of the number of bifidobacteria was accompanied by a significant drop of activity of total fecal β-galactosidase.

Studies that were conducted had demonstrated that preparations containing 5-ASA or a substance that breaks down in the body and forms 5-ASA demonstrate their anti-inflammatory activity after microbial transformation in the large intestine. In this, the released 5-ASA becomes active only as a result of bacterial breakdown.

In addition, microflora imbalance in the large intestine plays an important role in pathogenesis of nonspecific ulcerative colitis and Crohn's disease, and the role of immune disbalances in such diseases has been proven. Bifidobacteria and lactobacteria that are used are natural and indispensable intestine inhabitants, non-toxic, non-virulent and non-toxigenic for people and animals. They have high antagonistic activity towards pathogenic and opportunistic microorganisms, inhibit their adhesion to the mucous membrane of the intestine, promote normalization of the gastrointestinal tract activity and body's increased nonspecific resistance, promote parietal digestion, synthesize aminoacids and polyvitamins, and have an immunomodulatory effect. According to clinical tests data, high doses of bifidobacteria (in particular, probifora and bifidumbacterin preparations), as well as lactobacteria, or mixtures thereof have a marked effect on reparation processes in the large intestine mucous membrane and promote marked fading of inflammation in it. The use of these bacteria promotes fast restoration of intestine microbiocenisis and the resulting improvement of parietal digestion and absorption processes, and improves immune system functions.

The joint use of normoflora and 5-ASA makes it possible to substantially reduce general toxic and allergic manifestations and promotes stabilization and restoration of natural intestine microflora, which in turn leads to increased 5-ASA efficiency due to synchronous microbial transformation (acetylation) of the latter.

Contents of 5-ASA or a substance that breaks down in the body and forms 5-ASA in the range of 0.05-1.00 g corresponds to a therapeutic dose that is usually prescribed for patients for treatment ulcerative colitis or Crohn's disease. A less than 10⁵ CFU contents of a liophylically dried microbial mass of live bacteria does not produce a curative effect because, with intestine microenvironment out of balance, it does not provide proper colonization activity and antagonistic efficiency. A more than 10⁵ CFU contents of a liophylically dried microbial mass of live bacteria is impractical because it will result in unjustified waste of it and increase the cost of treatment.

The use of bacteria immobilized on a sorbent ensures high local colonization of, and increases restorative processes in, intestine mucous membranes. As a sorbent, one can use, in particular, fine activated charcoal or silicon dioxide.

As 5-ASA or a substance that breaks down in the body and forms 5-ASA, one can use preparations that contain mainly mesalazane or sulfasalazane. However, one can successfully use other aminosalicylates that are close to 5-ASA in chemical structure and have a similar mechanism of action, particularly, olsalazane, balsalazide, etc.

It is good practice to use mesalazane or sulfasalazane in powder form for preparing medical microclysters and for rectal administration, and in granular form for peroral administration. For convenient administration and to make it possible to dissolve preparation components in certain gastrointestinal tract areas, the components can be grouped into two capsules that together form a single dose, wherein one capsule contains a microbial mass of live bacteria, and the other capsule contains mesalazane or sulfasalazane. Alternately, the components can be grouped into two capsules, wherein one capsule contains a microbial mass of live bacteria and is located inside the second capsule that contains mesalazane or sulfasalazane.

The following examples illustrate the invention. Examples 1-6 characterize the composition of the claimed preparation.

### EXAMPLE 1

### 5-ASA, Powder, 0.05 g of Liophilized Mass of Live Bifidobacteria, CFU 5×10

### EXAMPLE 2

### 5-ASA, Powder, 0.10 g of Liophilized Mass of Live Bifidobacteria and Lactobacteria, CFU 10

### EXAMPLE 3

### 5-ASA, Powder, 0.25 g of Liophilized Mass of Live Sorbed Lactobacteria, CFU 10⁶

### EXAMPLE 4

### 5-ASA, Granules, 0.50 g of Liophilized Mass of Live Sorbed Bifidobacteria, CFU 5×10⁶

### EXAMPLE 5

### 5-ASA, Granules, 0.75 G of Liophilized Mass of Live Lactobacteria, CFU 10⁸

### EXAMPLE 6

### 5-ASA, Granules, 1.00 g of Liophilized Mass of Live Sorbed Bifidobacteria and Lactobacteria, CFU 5×10⁹

### EXAMPLE 7

### Assessment of Preparation's Safety in Animal Experiments

At the preclinical studies stage, assessment of safety of the proposed formulation and its individual components was conducted in experiments on white mice. For this purpose, a method used for medicinal immunobiologic preparations was selected. The study was conducted on mice with the mass not more than 15 g that, unlike adult mice, are characterized by hypersensitivity to the effect of alimentary factors. For five days, observations registered body mass changes and any symptoms of a disease. The body mass drop and appearance of symptoms of a disease or animal death indicate harmful action on mice organism.

The following substances were subjected to assessment for “safety”: the probiotic component of the preparation, containing bifidobacteria and lactobacteria in the amount of at least 10⁸ CFU, and a mixture of the probiotic component with sulfasalazane in doses of 50, 100 and 150 mg/kg of the animal body mass. The study results demonstrated that, taken separately, the probiotic component is safe when introduced directly to white mice stomach; the addition of sulfasalazane to the probiotic in the dose of 50 and 100 mg/kg had not affected the safety; increasing the sulfasalazane dose to 150 mg/kg made it possible to detect harmfulness symptoms (a 10% reduction of the mice body mass).

Thus, the preparation dose containing 100 mg of 5-ASA and at least 10 CFU of probiotics per kg of animal's body, which is higher than the human therapeutic daily dose, is well tolerated by animals. Examples 8-10 characterize preparation's efficiency in treating ulcerative colitis and Crohn's disease.

### EXAMPLE 8

Patient C, 28 years old.

Clinical diagnosis: Nonspecific ulcerative colitis, overall affection, high activity, relapsing form.

In the anamnesis: Duration of the disease—5 years. Hospitalization during the exacerbation period—40-60 days.

During examination, exacerbation of a moderately severe nonspecific ulcerative colitis was found. Clinically: stool frequency 4-6 times a day, moderate touch of blood; low-grade fever; no cardiovascular system pathology found, cardiac rate up to 90 beats per minute; ESR—30 mm/hour, moderate leukocytosis; insignificant malabsorption; intestinal pain at rest and during palpation.

During endoscopy: diffuse hyperemia; mucous granularity and edema observed; no vascular pattern; moderate bleeding; multiple erosions; isolated ulcers; fibrin; no pus. Based on the data of microbiological studies of feces—microbiocenosis imbalance: bifidobacteria content reduced to 10 CFU/g, lactobacteria content reduced to 10⁵ CFU/g, colon bacillus content reduced to 7×10⁶ CFU/g, cocci forms in the total microorganisms sum increased to 95% (the norm is not more than 25%). During complex treatment, the patient had been given for 21 days a preparation containing 4 g/day of 5-ASA and 5×10⁸ CFU of bifidobacteria on a sorbent, corresponding to example 4.

Before discharge after the administered therapy, positive dynamics was noticed—remission of nonspecific ulcerative colitis. Clinically: stool frequency per norm, stool without pathological admixtures; body temperature normal; no tachycardia; no intestinal pain. The positive dynamics of the endoscopic view: insignificant mucous membrane erythema; sponginess; diffused vascular pattern. C-reactive protein negative, hemoglobylin content and ESR are within normal limits.

Marked positive dynamics based on the data of microbiological studies of feces: normal bifidobacteria content (10⁸ CFU/g), normal lactobacteria content (10 CFU/g), increased colon bacillus content (10⁷ CFU/g) compared to data before treatment, reduced number of cocci forms in the total microorganism sum (50%).

Thus, ulcerative colitis exacerbation was arrested in 21 days. The patient was discharged in satisfactory condition.

Recommendation: take the preparation containing 5-ASA, while reducing the daily dose to 1 g, together with sorbed probiotic.

### EXAMPLE 9

Patient JI., 58 years old. Clinical diagnosis: nonspecific ulcerative colitis, distal form (chronic ulcerous proctitis), moderately severe course.

From the anamnesis: has had the disease for 28 years, with exacerbation and remission periods; always hospitalized during exacerbation; average hospital stay—up to 40 days. Hemocolitis is usually arrested during the 2nd or 3rd week of treatment.

During examination, exacerbation of a moderately severe nonspecific ulcerative colitis was found.

Clinically: stool frequency up to 6 times a day, with mucus and blood. Blood streaks in feces. Low-grade fever; abdominal pain; pain when defecating; moderately evident changes in the electrocardiogram, tachycardia up to 85 beats per minute. A 4 kg weight loss over the last six months is noticed. ESR 43 mm/hour, moderate leukocytosis; moderately evident malabsorption.

During endoscopy: diffuse hyperemia; evident mucus granularity and edema; no vascular pattern; moderate evident bleeding; multiple erosions; isolated ulcers; fibrin; insignificant amount of pus.

Marked positive dynamics based on the data of microbiological studies of feces—microbiocenosis imbalance: bifidobacteria content down to 10⁷ CFU/g, lactobacteria content down to 10⁵ CFU/g.

During complex treatment the patient was receiving a preparation corresponding to example 5 containing 5-ASA in the amount of 4 g/day and lactobacteria in the amount of 10⁸ CFU.

The characteristic feature of patient's treatment regimen was the fact that preparation 5-ASA was administered in a single step with lactobacteria.

The treatment course lasted 25 days. Hemocolitis was arrested on the 6^{th} day of treatment.

After the course, there is no intestinal pain, stool frequency is within normal limits, no pathological admixtures in stool, body temperature is normal, no tachycardia.

During endoscopy: insignificant mucous membrane erythema, diffused vascular pattern. ESR dropped down to 18 mm/hour. Based on the data of microbiological studies of feces, there are no symptoms of intestinal microcenosis imbalance: bifidobacteria content 10⁷ CFU/g, lactobacteria content 10 CFU/g.

The patient was discharged in satisfactory condition, with recommendation to continue outpatient treatment for 4 months, taking a 1 g/day dose of a 5-ASA-containing preparation and sorbed bifidobacteria.

### EXAMPLE 10

Patient P. 30 years old.

Diagnosis at admission: Crohn's disease.

Admitted to the hospital with body temperature under 37.5°; complained about sharp weakness, muscular tonus disturbance, muscular, abdominal and joint pain, diarrhea and weight loss.

In the anamnesis: no mention of digestive tract diseases, but suspicion of previous yersiniosis; because of this, the patient underwent comprehensive checkup for antibodies to various bacterial and viral infectious matter, including Yersinias, and campilobacteria, and to candidas. Digestive tract diseases were ruled out.

Colonoscopy and gastroscopy found gastritis, duodenitis, focal endocolitis, and dysbacteriosis.

During examination at the hospital: pale skin and mucous membranes; increased pulse rate; fever; bloating; moderate pain in the left iliac region; during profound abdominal palpation, the sigmoid colon is spasmodic. Diarrhea 4-5 times a day, shapeless stool with touch of blood, mucus and pus. Leukocytosis and increased ESR are noticed. A biochemical study recorded reduced concentration of potassium, sodium and magnesium in blood serum, increased creatine phosphokinase, and increased saturation percentage of iron, alpha and gamma globulins and beta 2 microglobulins. Low blood albumin.

A bacteriological study of feces for dysbacteriosis found reduced quantity of colon bacillus with normal functional activity, and enterococci, bifidobacteria and lactobacteria. Complex treatment was prescribed, using a preparation corresponding to example 6 containing 5-ASA in the amount of 4 g/day and 10 CFU of the mixture of sorbed bifidobacteria and lactobacteria—1 pack 3 times a day. Treatment course duration 21 days.

After the treatment, patient's condition improved significantly. Stool frequency—once a day, with no pathological admixtures; abdominal pain disappeared. A bacteriological study of feces for dysbacteriosis found evident positive dynamics. The number of bifidobacteria and lactobacteria, normal colon bacillus and enterococci had increased.

Thus, the claimed preparation is efficient in treatment of non-infectious inflammatory intestinal diseases and makes it possible to reduce the length of treatment.

### FORMULA OF INVENTION

1. A preparation for treatment of non-infectious inflammatory intestinal diseases containing 5-aminosalicylic acid or a substance that breaks down in the body and forms 5-aminosalicylic acid, distinct in that in addition it also contains a liophylically dried microbial mass of live bifidobacteria or lactobacteria, or a mixture thereof, with the following component ratio, g: 5-aminosalicylic acid or a substance that breaks down in the body and forms 5-aminosalicylic acid 0.05-1.00 a liophylically dried microbial mass of live bacteria 10⁵-10¹⁰ CFU.
2. A preparation per claim 1, distinct in that contains bifidobacteria, or bifidobacteria immobilized on a sorbent.
3. A preparation per claim 1, distinct in that contains lactobacteria, or lactobacteria immobilized on a sorbent.
4. A preparation per claim 1, distinct in that contains a mixture of bifidobacteria and lactobacteria, or a mixture of bifidobacteria and lactobacteria immobilized on a sorbent.
5. A preparation per claim 1, distinct in that, as 5-aminosalicylic acid or a substance that breaks down in the body and forms 5-aminosalicylic acid, it contains mesalazane or sulfasalazane.
6. A preparation per claim 5, distinct in that contains mesalazane or sulfasalazane in the form of powder or granules.
7. A preparation per either claim 1 or 6, distinct in that the components are grouped into two capsules that together form a single dose, wherein one capsule contains a microbial mass of live bacteria, and the other capsule contains mesalazane or sulfasalazane.
8. A preparation per either claim 1 or 6, distinct in that the components are grouped into two capsules, wherein one capsule contains a microbial mass of live bacteria and is located inside the second capsule that contains mesalazane or sulfasalazane.

What is claimed is:

1. A preparation for treatment of non-infectious inflammatory intestinal diseases containing 5-aminosalicylic acid or a substance that breaks down in the body and forms 5-aminosalicylic acid, distinct in that in addition it also contains a liophylically dried microbial mass of live bifidobacteria or lactobacteria, or a mixture thereof, with the following component ratio, g: 5-aminosalicylic acid or a substance that breaks down in the body and forms 5-aminosalicylic acid 0.05-1.00 a liophylically dried microbial mass of live bacteria 10⁵-10¹⁰ CFU.

2. A preparation per claim 1, distinct in that contains bifidobacteria, or bifidobacteria immobilized on a sorbent.

3. A preparation per claim 1, distinct in that contains lactobacteria, or lactobacteria immobilized on a sorbent.

4. A preparation per claim 1, distinct in that contains a mixture of bifidobacteria and lactobacteria, or a mixture of bifidobacteria and lactobacteria immobilized on a sorbent.

5. A preparation per claim 1, distinct in that, as 5-aminosalicylic acid or a substance that breaks down in the body and forms 5-aminosalicylic acid, it contains mesalazane or sulfasalazane.

6. A preparation per claim 5, distinct in that contains mesalazane or sulfasalazane in the form of powder or granules.

7. A preparation per either claim 1 or 6, distinct in that the components are grouped into two capsules that together form a single dose, wherein one capsule contains a microbial mass of live bacteria, and the other capsule contains mesalazane or sulfasalazane.

8. A preparation per either claim 1 or 6, distinct in that the components are grouped into two capsules, wherein one capsule contains a microbial mass of live bacteria and is located inside the second capsule that contains mesalazane or sulfasalazane.
